(19) United States
(12) Patent Application Publication
(10) Pub. No.: US 2024/0398215 A1
(43) Pub. Date: Dec. 5, 2024

(54) ELECTRODE-EQUIPPED ENDOSCOPE SYSTEM

(71) Applicant: HOYA CORPORATION, Tokyo (JP)

(72) Inventor: Kohei IKETANI, Tokyo (JP)

(73) Assignee: HOYA CORPORATION, Tokyo (JP)

(21) Appl. No.: 18/695,518

(22) PCT Filed: Dec. 20, 2022
(86) PCT No.: PCT/JP2022/046780

§ 371 Date: Mar. 26, 2024

(30) Foreign Application Priority Data
Jan. 7, 2022 (JP) 2022-001704

(51) Int. Cl. A61B 1/005; A61B 1/00; A61B 1/018; A61B 1/307

(52) U.S. Cl. CPC A61B 1/0051; A61B 1/00103; A61B 1/018; A61B 1/307

(57) ABSTRACT

Provided is an electrode-equipped endoscope system in which an insertion portion to be inserted into a body cavity can be freely bent. In an electrode-equipped endoscope system used for a surgical operation performed by inserting an external electrode into a body cavity, a recovery electrode, through which a current from the external electrode flows, is provided at a distal end portion of an insertion portion to be inserted into a bladder, and an active bending section is provided at the distal end portion of the insertion portion.

### TECHNICAL FIELD

The present invention relates to an electrode-equipped endoscope system including an insertion portion to be inserted into a body cavity.

The present application claims priority based on Japanese Patent Application No. 2022-001704 filed on Jan. 7, 2022, the entire contents of which are incorporated herein by reference.

### BACKGROUND ART

Conventional endoscopes that resect or coagulate tissues in a subject using a high-frequency current flowing from an electrode in an electrolyte solution environment have been widely used.

For example, Patent Literature 1 discloses a cystoscope in which an elastic region having lower flexural rigidity than a distal-end rigid portion provided at a distal end portion of an electrode support portion to be inserted into a subject and having an electrode and a proximal-end rigid portion connected with a proximal end of the electrode support portion is provided between the distal-end rigid portion and the proximal-end rigid portion.

### CITATION LIST

### Patent Literature

Patent Literature 1: WO 2020/136814 A

### SUMMARY OF INVENTION

### Technical Problem

A case is conceivable in which a lesion is generated in the vicinity of a communication port with the urethra in the bladder, and a surgical operation of such a lesion is performed. In such a case, since the lesion is located in the vicinity of the communication port with the urethra, it is necessary to perform a surgical operation in a state where a distal tip portion of the cystoscope is bent at an obtuse angle in a direction opposite to a direction in which the cystoscope enters the bladder, that is, in a state where the distal tip portion of the cystoscope forms an acute angle. In the case of a so-called rigid cystoscope, in which a bending operation of a distal tip portion cannot be performed, it is difficult to perform a surgical operation of a lesion at such a site.

However, in the cystoscope of Patent Literature 1, in which the electrode support portion has the elastic region, the distal-end rigid portion provided at the distal end portion of the electrode support portion and having the electrode can be deformed to such an extent that the distal-end rigid portion is slightly warped by contact with an inner wall of the bladder, but the electrode support portion or the distal-end rigid portion cannot be largely bent. Thus, the above-described problem cannot be solved.

The present invention has been made in view of such circumstances, and an object thereof is to provide an electrode-equipped endoscope system used for a surgical operation performed using an external electrode, in which an insertion portion to be inserted into a body cavity can be freely bent.

### Solution to Problem

An electrode-equipped endoscope system according to the present invention is an electrode-equipped endoscope system used for a surgical operation performed using an external electrode, the electrode-equipped endoscope system including: a corresponding electrode provided at a distal end portion of an insertion portion to be inserted into a body cavity and through which a current from the external electrode flows; and a bending section provided at the distal end portion.

In the present invention, a user bends a distal tip portion of the insertion portion by operating the bending section, and performs a surgical operation to resect or coagulate a lesion using the external electrode. At this time, a current from the external electrode flows through the corresponding electrode to be recovered.

### Advantageous Effects of Invention

According to the present invention, it is possible to provide an electrode-equipped endoscope system in which an insertion portion to be inserted into a body cavity can be freely bent.

### BRIEF DESCRIPTION OF DRAWINGS

FIG. 1 is an external view of an electrode-equipped endoscope system according to a first embodiment of the present invention.

FIG. 2 is a drawing illustrating a distal tip surface of an image capturing unit of the electrode-equipped endoscope system of the first embodiment.

FIG. 3 is a cross-sectional view taken along line III-III in FIG. 2.

FIG. 4 is a cross-sectional view schematically illustrating a configuration of an imaging device of the electrode-equipped endoscope system of the first embodiment.

FIG. 5 is a perspective view for explaining a state where a recovery electrode is provided in an active bending section of the electrode-equipped endoscope system of the first embodiment.

FIG. 6 is a cross-sectional view taken along line A-A in FIG. 5.

FIG. 7 is a perspective view illustrating a high-frequency knife used in the electrode-equipped endoscope system of the first embodiment.

FIG. 8 is an exemplary view for explaining a surgical operation using the electrode-equipped endoscope system of the first embodiment.

FIG. 9 is a perspective view for explaining a state where a recovery electrode is provided on an outer surface of a flexible portion on an operation unit side with respect to an active bending section in an electrode-equipped endoscope system of a second embodiment.

FIG. 10 is a perspective view for explaining a state where a recovery electrode is provided on an outer surface of a flexible portion on an operation unit side with respect to an active bending section in an electrode-equipped endoscope system of a third embodiment.

FIG. 11 is a perspective view for explaining a state where a recovery electrode is provided on an outer surface of a flexible portion on an operation unit side with respect to an active bending section in an electrode-equipped endoscope system of a fourth embodiment.

FIG. 12 is a perspective view for explaining a state where a recovery electrode is provided on an outer surface of a flexible portion on an operation unit side with respect to an active bending section in an electrode-equipped endoscope system of a fifth embodiment.

### DESCRIPTION OF EMBODIMENTS

Hereinafter, an electrode-equipped endoscope system according to embodiments of the present invention will be described in detail with reference to the drawings.

### First Embodiment

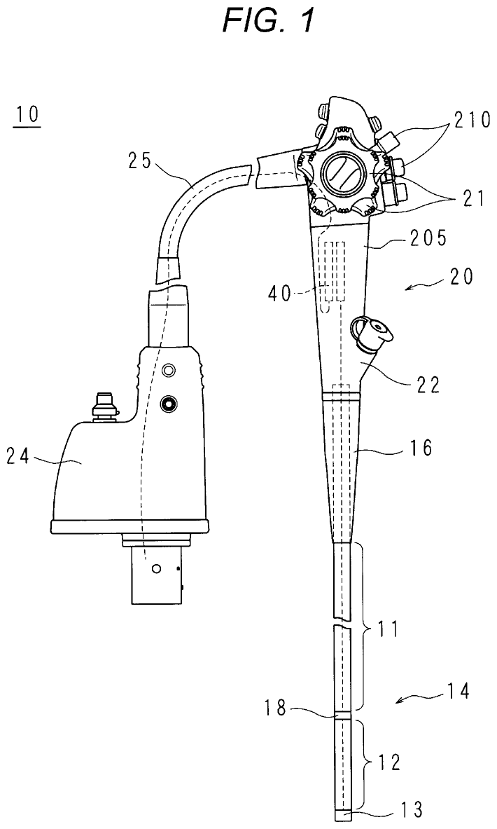

FIG. 1 is an external view of an electrode-equipped endoscope system 10 according to a first embodiment of the present invention. The electrode-equipped endoscope system 10 of the present embodiment includes a flexible disposable endoscope, and is used in a case where a surgical operation such as resection is performed using a so-called high-frequency knife in an electrolyte solution such as physiological saline in a state where the inside of the bladder is filled with such an electrolyte solution (in an electrolyte solution environment). For example, the electrode-equipped endoscope system 10 is for single use.

The electrode-equipped endoscope system 10 includes an insertion portion 14 to be inserted into the bladder of a subject, an operation unit 20 for operating the insertion portion 14, and a connector unit 24 connected with a processor or the like, which are not illustrated.

The insertion portion 14 is connected with the operation unit 20 through a bend preventing portion 16, and the connector unit 24 is connected with the operation unit 20 through a universal cord 25.

The universal cord 25 has pliability, and includes an electric line that sends an electric signal from an imaging means of the insertion portion 14 to the connector unit 24, a flow passage through which the electrolyte solution passes, and the like.

The operation unit 20 includes a grip portion 205, a button 210 for receiving an instruction from a user, and a bending knob 21 for operating a bending motion of an active bending section 12 to be described later.

The grip portion 205 has a substantially cylindrical shape, and a channel inlet 22, through which a treatment tool such as the high-frequency knife is to be inserted into the bladder from outside, is provided therein near the bend preventing portion 16. In addition, a relay portion 40, which connects an imaging unit 617 of an image capturing unit 13 to be described later with the processor, is incorporated inside the grip portion 205.

The insertion portion 14 has a cylindrical shape with a small diameter, and is configured to be freely bendable. The insertion portion 14 includes the image capturing unit 13, the active bending section 12, and a flexible portion 11 sequentially in this order from the distal tip side. The flexible portion 11 is connected with the active bending section 12 through a connection portion 18.

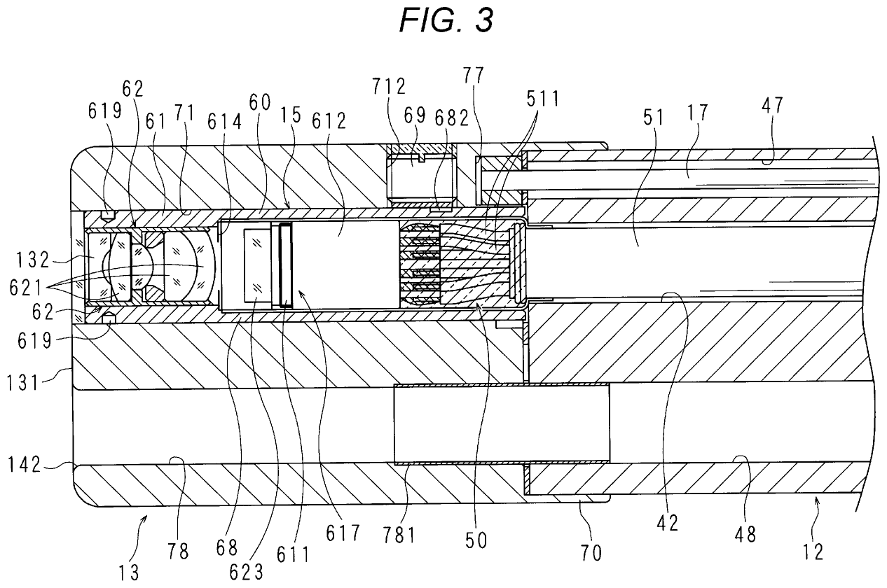

The image capturing unit 13 includes the imaging unit 617 including an imaging means such as a charge coupled device (CCD) or a complementary metal oxide semiconductor (CMOS), a circuit board for driving the imaging means, an observation optical system, and the like, and a lens unit 62 including a series of lens sets (see FIG. 3). In addition, the image capturing unit 13 includes an illumination optical system or the like for irradiating an observation target site in the bladder with light. An electric signal from the imaging unit 617 of the image capturing unit 13 is sent to the processor through the relay portion 40 and the connector unit 24.

The active bending section 12 is actively bendable. That is, the active bending section 12 is freely bent in four directions in accordance with an operation of the bending knob 21. On the other hand, the flexible portion 11 is passively bent. That is, the flexible portion 11 is bent by contact with an object.

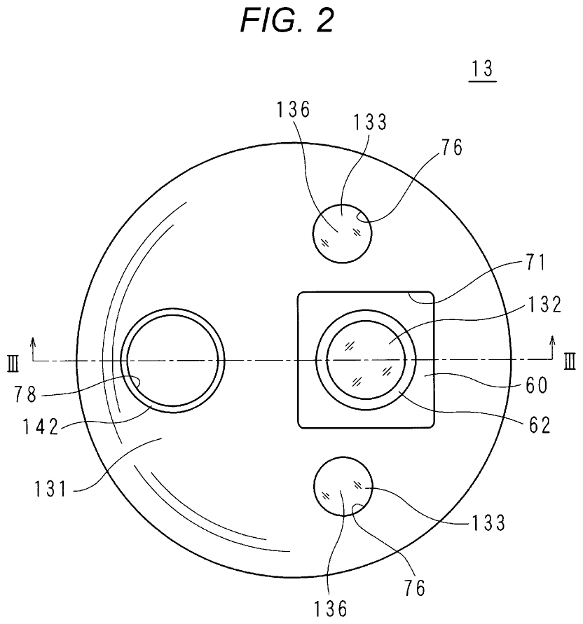

FIG. 2 is a drawing illustrating a distal tip surface 131 of the image capturing unit 13 of the electrode-equipped endoscope system 10 of the first embodiment, and FIG. 3 is a cross-sectional view taken along line III-III in FIG. 2.

The image capturing unit 13 has a columnar shape, and has a diameter slightly larger than that of the active bending section 12. In the image capturing unit 13, a flange portion 70 is provided along the circumferential edge on an end surface on the active bending section 12 side. The distal end portion of the active bending section 12 is internally fit with the flange portion 70.

A unit hole 71, which penetrates in the axial length direction, is formed in the image capturing unit 13. The unit hole 71 is opened at a substantially central portion of the circular distal tip surface 131, and has a quadrangular shape in a cross-sectional view, and an imaging device 15 is inserted therein.

In the electrode-equipped endoscope system 10, the imaging device 15 is incorporated in the insertion portion 14 and the bend preventing portion 16. The imaging device 15 includes the lens unit 62 and the imaging unit 617, and the imaging unit 617 is connected with the relay portion 40 through a cable unit 50.

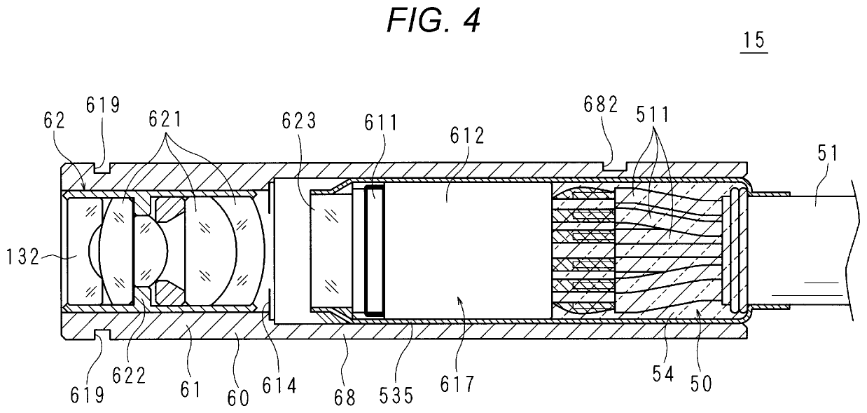

FIG. 4 is a cross-sectional view schematically illustrating a configuration of the imaging device 15 of the electrode-equipped endoscope system 10 of the first embodiment.

The imaging device 15 includes a housing tube 60, and houses the lens unit 62 and the imaging unit 617. The housing tube 60 is a quadrangular tube, one end portion is a circular hole portion 61 having a circular inner circumferential surface, and the other part is a quadrangular hole portion 68 having a quadrangular inner circumferential surface. The lens unit 62 is inserted into the circular hole portion 61, and the imaging unit 617 is inserted into the quadrangular hole portion 68. A light shielding mask 614 is provided between the lens unit 62 and the imaging unit 617.

The lens unit 62 includes an observation window 132 and a plurality of imaging lenses 621. The observation window 132 is exposed to the distal tip surface 131, and the imaging lenses 621 are provided on the back side of the observation window 132. The observation window 132 and the plurality of imaging lenses 621 are coaxially disposed. Outer circumferential surfaces of the observation window 132 and the plurality of imaging lenses 621 are covered with and secured by a lens frame 622.

The imaging unit 617 includes a filter 623, an image sensor 611, and an imaging board 612.

The filter 623 removes unnecessary light such as infrared rays from the light that enters the image sensor 611. The light that enters from the lens unit 62 forms an image on the image sensor 611, and the image sensor 611 converts an optical image into an electric signal. In the imaging board 612, a driver circuit that controls the image sensor 611 is embedded. The cable unit 50 is connected with the imaging board 612 from the other end side of the housing tube 60.

The cable unit 50 includes a plurality of cable strands 511 bundled by a cable tube 51. One end of the cable unit 50 is connected with the imaging board 612. A connection between one end of the cable unit 50 and the imaging board 612 is potted with an insulating resin 54.

The filter 623, the image sensor 611, the imaging board 612, and the one end portion of the cable unit 50 are held, in an assembled state, by an insulating tube 535.

The cable unit 50 extends outward from the other end of the housing tube 60. The other end of the cable unit 50 is connected with the relay portion 40 through the active bending section 12 and the flexible portion 11.

In the housing tube 60, at an end portion of the circular hole portion 61, claw holes 619 are respectively formed on outer surfaces of two side walls facing each other. In addition, a recess 682 is formed on one of the outer surfaces of the two side walls, at an end portion of the quadrangular hole portion 68 (see FIG. 4). The side wall, on which the recess 682 and the claw hole 619 are formed, is located closer to an outer circumferential surface of the image capturing unit 13 than the other three side walls.

A cable hole 42 is formed at a position corresponding to the unit hole 71, in the active bending section 12 (see FIG. 3). The cable hole 42 has a circular shape in a cross-sectional view, and penetrates the active bending section 12 in the axial length direction. The cable hole 42 of the active bending section 12 communicates with the unit hole 71 of the image capturing unit 13, and the cable unit 50 is inserted into the cable hole 42. The cable unit 50 extends, through the flexible portion 11 and the bend preventing portion 16, to the relay portion 40 of the operation unit 20.

Although not illustrated, the active bending section 12 has a bending mechanism in which a plurality of pieces, which are metal annular members, are connected in the axial length direction. A user can operate the bending knob 21 of the operation unit 20 to control the bending of the bending mechanism and bend the active bending section 12 in a predetermined direction.

In addition, an entire region of outer circumferential surfaces of the pieces is covered with a metal blade 122 formed by knitting an extra-fine metal yarn. Furthermore, the outer side of the metal blade 122 is covered with an insulating resin material 121 (see FIG. 6).

As illustrated in FIG. 2, in the distal tip surface 131 of the image capturing unit 13, an illumination window 133 is provided on each of opposite sides of the observation window 132. An illumination hole 76, into which a light emitting element 136 is inserted, penetrates the image capturing unit 13 in the axial length direction. The illumination hole 76 has a circular shape in a cross-sectional view, and has one end opened on the distal tip surface 131, and the illumination window 133 is provided at such one end. The illumination window 133 expands an irradiation angle of the illumination light emitted from the light emitting element 136, and causes the illumination light to emit.

A bending wire hole for a bending wire 17, which penetrates the insertion portion 14 in the axial length direction, and which is for an operation of bending the image capturing unit 13 (the active bending section 12), is formed in the insertion portion 14. The bending wire hole includes a bending wire hole 47 formed in the active bending section 12 and a bending wire hole (not illustrated) formed in each of the flexible portion 11 and the bend preventing portion 16. For convenience, only the bending wire hole 47 will be described in the following.

The bending wire hole 47 has a circular shape in a cross-sectional view, and four bending wire holes 47 are formed in the vicinity of an outer circumferential surface of the active bending section 12. The bending wire holes 47 are formed to be separated at equal intervals in the circumferential direction of the active bending section 12. One ends of the bending wire holes 47 are opened on an end surface of the active bending section 12, on the image capturing unit 13 side.

In addition, in the image capturing unit 13, a recess 77 is formed at a position corresponding to such one end of each of the bending wire holes 47, on the end surface on the active bending section 12 side. Each recess 77 has a circular shape in a cross-sectional view, and has a diameter larger than that of the bending wire hole 47.

The bending wire 17 is inserted into each bending wire hole 47. One end of the bending wire 17 protrudes into the corresponding recess 77 through such one end of the bending wire hole 47. One end of the bending wire 17 is secured in the recess 77 by brazing, caulking, or the like. The other end of the bending wire 17 is connected with the bending knob 21 of the operation unit 20 through the bending wire hole 47. The user operates the bending knob 21 to cause the bending wire 17 to bend the active bending section 12.

In addition, as illustrated in FIG. 3, on the outer circumferential surface of the image capturing unit 13, a communication hole 712, which communicates with the unit hole 71, is formed at a position corresponding to the recess 682 of the housing tube 60 in the radial direction. The communication hole 712 has a circular shape in a cross-sectional view, and a screw thread is formed on its inner circumferential surface. A securing screw 69 is screwed into the communication hole 712. When the securing screw 69 comes into pressure contact with the housing tube 60, the position of the housing tube 60 in the unit hole 71 is secured.

In the insertion portion 14, a channel, which penetrates the insertion portion 14 in the axial length direction, and through which the high-frequency knife that has been inserted from the channel inlet 22 passes, is formed. The channel includes a channel 78 formed in the image capturing unit 13, a channel 48 formed in the active bending section 12, and a channel (not illustrated) formed in the flexible portion 11.

One end of the channel 78 is enlarged in diameter to form a forceps port 142, and is opened in the vicinity of the observation window 132 on the distal tip surface 131. The other end of the channel 78 communicates with one end of the channel 48 through a channel pipe 781 (see FIG. 3).

That is, the channel pipe 781 is provided across the image capturing unit 13 and the active bending section 12. One end of the channel pipe 781 is internally fit with the channel 78, and the other end of the channel pipe 781 is internally fit with the channel 48.

In the electrode-equipped endoscope system 10 of the first embodiment, a recovery electrode 19 (corresponding electrode) that recovers a current flowing from the high-frequency knife through the electrolyte solution is provided in the active bending section 12. That is, the recovery electrode 19 is provided integrally with the insertion portion 14.

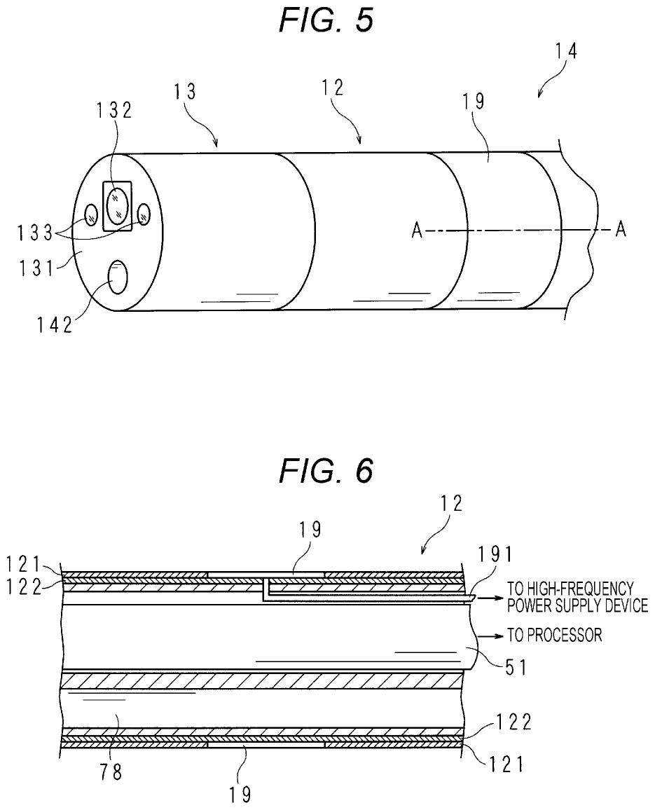

FIG. 5 is a perspective view for explaining a state where the recovery electrode 19 is provided in the active bending section 12 of the electrode-equipped endoscope system 10 of the first embodiment, and FIG. 6 is a cross-sectional view taken along line A-A in FIG. 5.

The recovery electrode 19 is made of, for example, a conductive metal such as stainless steel, and has a cylindrical shape. The recovery electrode 19 is externally fit with a recess provided on the outer circumferential surface of the active bending section 12 such that its outer diameter is substantially equal to the outer diameter of the active bending section 12 and its axis coincides with the axis of the active bending section 12. An outer circumferential surface of the recovery electrode 19 is flush with the resin material 121 of the active bending section 12 so as not to hinder the insertion of the electrode-equipped endoscope system 10 into the bladder. In addition, the recovery electrode 19 is insulated from the metal blade 122.

A connection line 191 is connected to an inner circumferential surface of the recovery electrode 19. The connection line 191 extends through the flexible portion 11 and the bend preventing portion 16, and electrically connects the recovery electrode 19 and a high-frequency power supply device together. For example, the connection line 191 may be configured to pass through the bending wire hole 47.

The flexible portion 11 is configured to have different hardness depending on the position in the axial length direction. One end portion of the flexible portion 11 on the active bending section 12 side is bendable, and the hardness of a portion other than such one end portion is configured to be higher than the hardness of the one end portion. Specifically, in a case where the bladder of a human is assumed to have a spherical shape with a diameter of 10 cm, the hardness of a 30 cm portion on the operation unit 20 side of the flexible portion 11 having a dimension of 40 cm in the axial length direction may be increased in view of the fact that the male urethra is said to have a length of 20 cm. That is, in the flexible portion 11, a ¾ portion on the operation unit 20 side has higher hardness than the remaining ¼ portion. In addition, in a boundary portion between these two portions, it is desirable that the hardness gradually changes rather than changing stepwise.

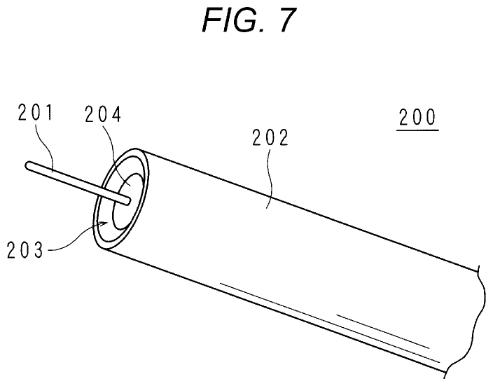

FIG. 7 is a perspective view illustrating a high-frequency knife 200 used in the electrode-equipped endoscope system 10 of the first embodiment. The high-frequency knife 200 is connected with the high-frequency power supply device.

The high-frequency knife 200 includes an elongated hose-shaped tube 202, a support portion 204 provided inside one end portion of the tube 202 and having a diameter smaller than the inner diameter of the tube 202, and a round bar-shaped electrode 201 (external electrode) provided in the support portion 204 and protruding outward through an opening on one end side of the tube 202. The high-frequency knife 200 can be freely bent. The round bar-shaped electrode 201 is provided on the front side of the support portion 204, and a coated conductive wire (not illustrated) for connecting the round bar-shaped electrode 201 and the high-frequency power supply device together is connected with the back side of the support portion 204. A gap 203 is formed between the tube 202, and the support portion 204 and the conductive wire.

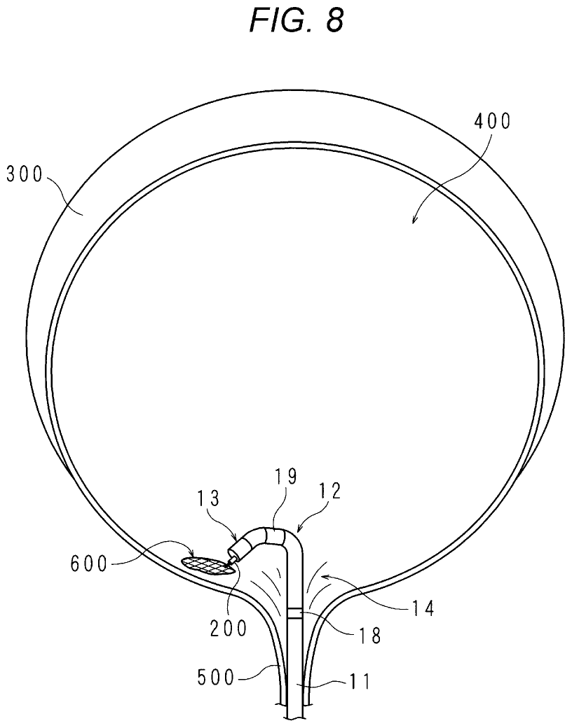

The electrolyte solution is supplied into a bladder 300 through the gap 203 (see FIG. 8). When the inside of the bladder 300 is filled with an electrolyte solution 400, the high-frequency knife 200 is inserted into the bladder 300 through the channel of the electrode-equipped endoscope system 10. When a high-frequency current is output from the high-frequency power supply device, the tissues of a subject in contact with the round bar-shaped electrode 201 generate heat, so that such heat is used to perform a surgical operation to resect or coagulate the tissues. The present invention is not limited to this, and the electrolyte solution may be supplied through the channel of the electrode-equipped endoscope system 10.

An operation of the electrode-equipped endoscope system 10 of the first embodiment having the above configuration will be described.

FIG. 8 is an exemplary view for explaining a surgical operation using the electrode-equipped endoscope system 10 of the first embodiment. For example, an operator inserts the electrode-equipped endoscope system 10 into the bladder 300 through a urethra 500 of a subject (male). At this time, the active bending section 12 and a part of the flexible portion 11 are inserted into the bladder 300. Next, the operator inserts the high-frequency knife 200 into the channel from the channel inlet 22 until the high-frequency knife 200 reaches the image capturing unit 13 of the electrode-equipped endoscope system 10. For example, the electrolyte solution is supplied into the bladder 300 from the high-frequency knife 200, and a surgical operation such as transurethral resection (TUR) is performed in a state where the inside of the bladder 300 is filled with the electrolyte solution 400.

When the high-frequency power supply device outputs a high-frequency current to the high-frequency knife 200, such a high-frequency current flows through the round bar-shaped electrode 201 of the high-frequency knife 200, the electrolyte solution 400, and the recovery electrode 19. That is, the recovery electrode 19 recovers the current from the round bar-shaped electrode 201 through the electrolyte solution 400. At this time, the tissues of the subject in contact with the round bar-shaped electrode 201 generate heat, which makes it possible to resect the tissues as a lesion or the like.

Here, as illustrated in FIG. 8, a case is conceivable in which a lesion 600 as a surgical operation target is located in the vicinity of a communication port with the urethra 500 in the bladder 300. In a case where the lesion 600 is generated in a region close to an opening site of the urethra 500 as described above, it is necessary to perform a surgical operation in a state where a distal tip portion of the insertion portion 14 is largely bent in a direction opposite to the entry direction of the insertion portion 14 into the bladder 300. Therefore, in the case of a so-called rigid cystoscope, in which a distal tip portion cannot be bent, it is difficult to perform a surgical operation of the lesion 600 in the vicinity of the urethra 500. Furthermore, the shape of the human urethra 500 is not linear. Thus, in a case where the rigid cystoscope, which is linear and cannot be bent, is used, the electrode-equipped endoscope system 10 (the insertion portion 14) gives a great pain to the subject when passing through the urethra 500.

On the other hand, in the electrode-equipped endoscope system 10 of the first embodiment, the distal tip portion of the insertion portion 14 including the active bending section 12 can be freely bent, and one end portion of the flexible portion 11 on the active bending section 12 side is also bendable.

Therefore, even in a case where the lesion 600 is generated in the vicinity of the communication port with the urethra 500 in the bladder 300, in the electrode-equipped endoscope system 10 of the first embodiment, the active bending section 12 can be bent such that the distal tip portion of the insertion portion 14 is bent at an obtuse angle to form an acute angle, that is, the distal tip surface 131 of the image capturing unit 13 faces the lesion 600.

Thereafter, the operator pushes out the high-frequency knife 200 from the forceps port 142 of the distal tip surface 131 to bring it closer to the lesion 600, and brings the round bar-shaped electrode 201 into contact with the lesion 600. As a result, resection or coagulation of the lesion 600 is performed by the current from the round bar-shaped electrode 201. In addition, the recovery electrode 19 recovers the current from the round bar-shaped electrode 201 through the electrolyte solution 400. Therefore, a surgical operation can be appropriately performed on the lesion 600 in the vicinity of the urethra 500.

In addition, the active bending section 12 (the distal tip portion of the insertion portion 14) and one end portion of the flexible portion 11 on the active bending section 12 side are bendable. Thus, when the electrode-equipped endoscope system 10 (the insertion portion 14) passes through the urethra 500, the active bending section 12 and the flexible portion 11 can be deformed according to the shape of the urethra 500, which makes it possible to reduce the pain of the subject.

In addition, in the electrode-equipped endoscope system 10 of the first embodiment, the imaging device 15 can be collected by removing the securing screw 69, pushing the recess 682 toward the distal tip side of the image capturing unit 13 using a jig to expose the claw holes 619, hooking a claw jig into the two claw holes 619, and pulling the claw jig. Therefore, environmental pollution and resource waste can be reduced.

In addition, in the electrode-equipped endoscope system 10 of the first embodiment, since the recovery electrode 19 is provided integrally with the insertion portion 14 as described above, it is not necessary to separately provide a counter electrode for recovering the current from the round bar-shaped electrode 201, and thus, it is possible to reduce the cost, and to prevent burns due to a contact failure occurring in the use of the counter electrode.

In addition, since the electrode-equipped endoscope system 10 of the first embodiment is a flexible cystoscope for single use as described above, there occur no contamination and contact failure of the recovery electrode 19 due to multiple uses, and there occurs no deterioration of the recovery electrode 19 due to cleaning of the electrode-equipped endoscope system 10.

Furthermore, in the electrode-equipped endoscope system 10 of the first embodiment, since the recovery electrode 19 has a cylindrical shape as described above, there is no limitation on the direction in recovering the current from the high-frequency knife 200 through the electrolyte solution, which can improve operability.

As described above, in the flexible portion 11, the hardness of the portion other than one end portion on the image capturing unit 13 side is configured to be higher than the hardness of the one end portion. Therefore, in the electrode-equipped endoscope system 10 of the first embodiment, a force is easily transmitted to the image capturing unit 13 of the electrode-equipped endoscope system 10 at the time of an operation such as twisting, which can improve the operability of the electrode-equipped endoscope system 10.

In the above description, a case where the recovery electrode 19 is made of a conductive metal, has a cylindrical shape, and is externally fit with the outer circumferential surface of the active bending section 12 is described as an example. However, the shape of the recovery electrode 19 is not limited to the cylindrical shape. For example, the recovery electrode 19 may have a pad shape and may be provided on a part of the outer circumferential surface of the active bending section 12. In addition, the recovery electrode 19 is not limited to the conductive metal, and may be made of conductive fiber.

Although a case where the recovery electrode 19 is provided in the active bending section 12 is described above as an example, the present invention is not limited to this. The recovery electrode 19 may be provided on the distal tip side with respect to the active bending section 12, or may be provided on the operation unit 20 side with respect to the active bending section 12, for example, on the connection portion 18. The recovery electrode 19 is placed in the electrolyte solution environment. If the recovery electrode 19 is too close to the distal tip surface 131, the current from the high-frequency knife 200 may directly flow to the recovery electrode 19 without passing through such a lesion. Therefore, the operation unit 20 side with respect to the active bending section 12 is more desirable than the distal tip side with respect to the active bending section 12.

### Second Embodiment

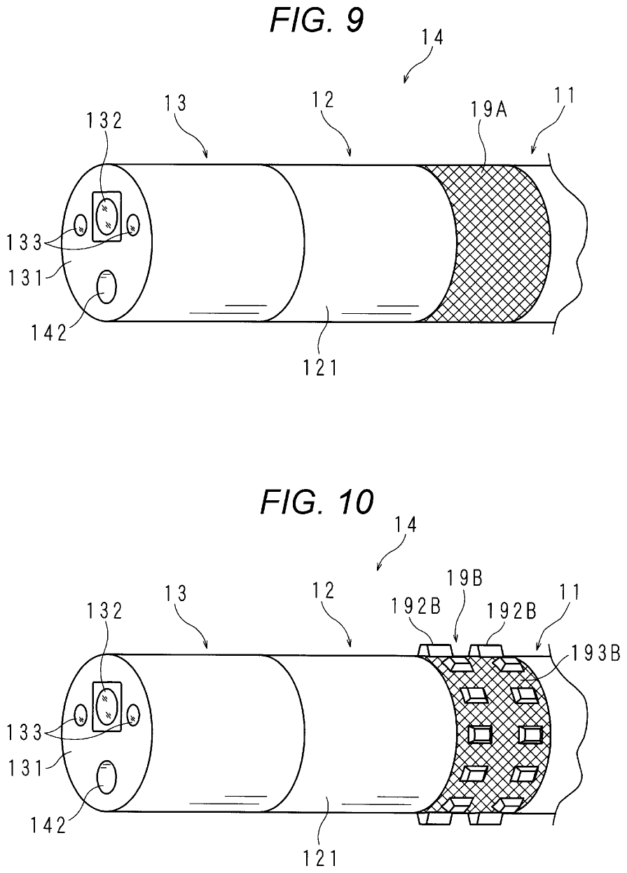

FIG. 9 is a perspective view for explaining a state where a recovery electrode 19A (corresponding electrode) is provided on an outer surface of the flexible portion 11 on the operation unit 20 side with respect to the active bending section 12 in the electrode-equipped endoscope system 10 of a second embodiment.

In the electrode-equipped endoscope system 10 of the second embodiment, the recovery electrode 19A is formed of, for example, a mesh-shaped conductive metal material and has a cylindrical shape. The recovery electrode 19A is externally fit with an outer circumferential surface of the flexible portion 11 such that its outer diameter is substantially equal to the outer diameter of the flexible portion 11 and its axis coincides with the axis of the flexible portion 11. An outer circumferential surface of the recovery electrode 19A is substantially flush with the resin material 121 of the flexible portion 11.

Similarly to the first embodiment, the connection line 191 for electrically connecting the recovery electrode 19A and the high-frequency power supply device together is connected with an inner circumferential surface of the recovery electrode 19A (see FIG. 6).

In the electrode-equipped endoscope system 10 of the second embodiment, since the recovery electrode 19A is made of a mesh-shaped conductive metal material as described above, the hindrance by the recovery electrode 19A is reduced in the bending motion of the active bending section 12, so that the active bending section 12 can be easily bent.

Other configurations of the electrode-equipped endoscope system 10 in the second embodiment are the same as those of the electrode-equipped endoscope system 10 in the first embodiment, and the same components as those of the first embodiment are denoted by the same reference numerals, and detailed descriptions will be omitted.

### Third Embodiment

FIG. 10 is a perspective view for explaining a state where a recovery electrode 19B (corresponding electrode) is provided on an outer surface of the flexible portion 11 on the operation unit 20 side with respect to the active bending section 12 in the electrode-equipped endoscope system 10 of a third embodiment.

In the electrode-equipped endoscope system 10 of the third embodiment, the recovery electrode 19B is formed of, for example, a mesh-shaped conductive metal material and includes a cylindrical portion 193B having a cylindrical shape and a plurality of protrusions 192B provided protruding from an outer circumferential surface of the cylindrical portion 193B. The cylindrical portion 193B is externally fit with an outer circumferential surface of the flexible portion 11 such that its outer diameter is substantially equal to the outer diameter of the flexible portion 11 and its axis coincides with the axis of the flexible portion 11. An outer circumferential surface of the cylindrical portion 193B is substantially flush with the outer circumferential surface of the flexible portion 11. Similarly to the first embodiment, the connection line 191 (see FIG. 6) for electrically connecting the recovery electrode 19B and the high-frequency power supply device together is connected with an inner circumferential surface of the recovery electrode 19B.

The plurality of protrusions 192B are made of, for example, a conductive metal such as stainless steel, and are disposed side by side at the same intervals in the circumferential direction of the cylindrical portion 193B to form a line. For example, there are two lines of the protrusions 192B, and the respective lines are disposed side by side at a predetermined interval in the axial direction of the cylindrical portion 193B (the flexible portion 11).

In the electrode-equipped endoscope system 10 of the third embodiment, since the cylindrical portion 193B of the recovery electrode 19B is made of a mesh-shaped conductive metal material as described above, the hindrance by the recovery electrode 19B (the cylindrical portion 193B) is reduced in the bending motion of the active bending section 12, so that the active bending section 12 can be easily bent.

Moreover, since the plurality of protrusions 192B are provided protruding on the outer circumferential surface of the cylindrical portion 193B, a gap is formed between an inner wall of the bladder 300 and the cylindrical portion 193B at the time of contact between the recovery electrode 19B and the inner wall of the bladder 300. Therefore, the electrolyte solution 400 can move through such a gap, and the electrolyte solution 400 and the recovery electrode 19B are reliably brought into contact with each other.

Other configurations of the electrode-equipped endoscope system 10 in the third embodiment are the same as those of the electrode-equipped endoscope system 10 in the first embodiment, and the same components as those of the first embodiment are denoted by the same reference numerals, and detailed descriptions will be omitted.

### Fourth Embodiment

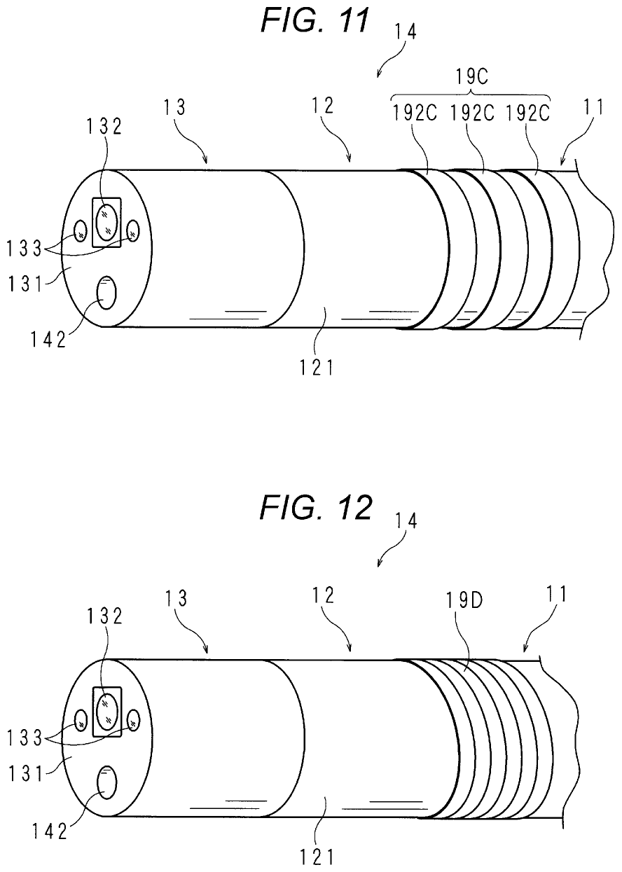

FIG. 11 is a perspective view for explaining a state where a recovery electrode 19C (corresponding electrode) is provided on an outer surface of the flexible portion 11 on the operation unit 20 side with respect to the active bending section 12 in the electrode-equipped endoscope system 10 of a fourth embodiment.

In the electrode-equipped endoscope system 10 of the fourth embodiment, the recovery electrode 19C includes a plurality of ring portions 192C made of a conductive metal material such as stainless steel, for example. In the fourth embodiment, a case where the recovery electrode 19C includes three ring portions 192C will be described as an example, but the present invention is not limited to this.

The plurality of ring portions 192C are provided at equal intervals in the axial direction of the flexible portion 11. In a case where the diameter of the image capturing unit 13 is 5 mm, the dimension of each of the ring portions 192C in the axial direction is, for example, 1 mm, and a ratio of the ring portion 192C to an interval between the ring portions 192C is, for example, 1:1 to 1:3.

Each of the ring portions 192C is externally fit with an outer circumferential surface of the flexible portion 11 such that its outer diameter is substantially equal to the outer diameter of the flexible portion 11 and its axis coincides with the axis of the flexible portion 11. An outer peripheral surface of each of the ring portions 192C is substantially flush with the outer circumferential surface of the flexible portion 11. Similarly to the first embodiment, the connection line 191 (see FIG. 6) for electrically connecting the recovery electrode 19C and the high-frequency power supply device together is connected with an inner circumferential surface of each of the ring portions 192C.

In the electrode-equipped endoscope system 10 of the fourth embodiment, since the recovery electrode 19C includes the plurality of ring portions 192C as described above, the hindrance by the recovery electrode 19C is reduced in the bending motion of the active bending section 12, so that the active bending section 12 can be easily bent.

The electrode-equipped endoscope system 10 of the fourth embodiment is not limited to the description above. For example, each of the ring portions 192C may be formed of a mesh-shaped conductive metal material, or may include the plurality of protrusions 192B (see FIG. 10).

Other configurations of the electrode-equipped endoscope system 10 in the fourth embodiment are the same as those of the electrode-equipped endoscope system 10 in the first embodiment, and the same components as those of the first embodiment are denoted by the same reference numerals, and detailed descriptions will be omitted.

### Fifth Embodiment

FIG. 12 is a perspective view for explaining a state where a recovery electrode 19D (corresponding electrode) is provided on an outer surface of the flexible portion 11 on the operation unit 20 side with respect to the active bending section 12 in the electrode-equipped endoscope system 10 of a fifth embodiment.

In the electrode-equipped endoscope system 10 of the fifth embodiment, the recovery electrode 19D is made of a conductive metal material such as stainless steel, for example, and has a spiral shape. The recovery electrode 19D is externally fit with an outer circumferential surface of the flexible portion 11 such that its outer diameter is substantially equal to the outer diameter of the flexible portion 11 and its axis coincides with the axis of the flexible portion 11. An outer circumferential surface of the recovery electrode 19D is substantially flush with the outer circumferential surface of the flexible portion 11. The connection line 191 (see FIG. 6) for electrically connecting the recovery electrode 19D and the high-frequency power supply device together is connected with any position of an inner circumferential surface of the recovery electrode 19D.

In the electrode-equipped endoscope system 10 of the fifth embodiment, since the recovery electrode 19D has a spiral shape as described above, the hindrance by the recovery electrode 19D is reduced in the bending motion of the active bending section 12, so that the active bending section 12 can be easily bent. Moreover, a stress (stress) applied to the recovery electrode 19D is dispersed, so that the risk of disconnection/breakage can be reduced.

The electrode-equipped endoscope system 10 of the fifth embodiment is not limited to the description above, and the recovery electrode 19D may be formed of a mesh-shaped conductive metal material.

Other configurations of the electrode-equipped endoscope system 10 in the fifth embodiment are the same as those of the electrode-equipped endoscope system 10 in the first embodiment, and the same components as those of the first embodiment are denoted by the same reference numerals, and detailed descriptions will be omitted.

Technical features (constitutional requirements) that have been described in the first to fifth embodiments can be combined with one another, and a new technical feature can be formed with the combination.

It should be noted that the embodiments disclosed herein are illustrative in all respects and are not restrictive. The scope of the present invention is indicated by the scope of claims, not the above-described meaning, and is intended to include all modifications within the meaning and scope equivalent to the claims.

### REFERENCE SIGNS LIST

10 Electrode-equipped endoscope system
11 Flexible portion
12 Active bending section
13 Image capturing unit
14 Insertion portion
18 Connection portion
19, 19A, 19B, 19C, 19D Recovery electrode
20 Operation unit
131 Distal tip surface
142 Forceps port
192B Protrusion
192C Ring portion
200 High-frequency knife
201 Round bar-shaped electrode
300 Bladder
400 Electrolyte solution
500 Urethra
600 Lesion

1. An electrode-equipped endoscope system used for a surgical operation performed using an external electrode, the electrode-equipped endoscope system comprising:
a corresponding electrode provided at a distal end portion of an insertion portion to be inserted into a body cavity and through which a current from the external electrode flows; and
a bending section provided at the distal end portion.

2. The electrode-equipped endoscope system according to claim 1,
wherein the external electrode is used in a bladder in an electrolyte solution environment, and
the current from the external electrode flows to the corresponding electrode through the electrolyte solution.

3. The electrode-equipped endoscope system according to claim 1, further comprising
an operation unit configured to operate a bending motion of the bending section, wherein
the corresponding electrode is provided on an outer surface of the insertion portion at the bending section, a distal tip side with respect to the bending section, or the operation unit side with respect to the bending section.

4. The electrode-equipped endoscope system according to claim 1, wherein the corresponding electrode has a cylindrical shape, and is externally fit with an outer circumferential surface of the insertion portion.

5. The electrode-equipped endoscope system according to claim 1, wherein
the corresponding electrode includes a plurality of ring members, and
the plurality of ring members are externally fit with an outer circumferential surface of the insertion portion at a predetermined interval in a length direction of the insertion portion.

6. The electrode-equipped endoscope system according to claim 4, wherein the corresponding electrode has a mesh shape.

7. The electrode-equipped endoscope system according to claim 4, wherein the corresponding electrode includes a plurality of protrusions.

8. The electrode-equipped endoscope system according to claim 1, wherein the corresponding electrode has a spiral shape.

9. The electrode-equipped endoscope system according to claim 3, wherein in a flexible portion provided on the operation unit side with respect to the bending section, hardness of a portion other than an end portion on the bending section side is higher than hardness of the end portion.

10. The electrode-equipped endoscope system according to claim 1, wherein a forceps port through which the external electrode passes is provided on a distal tip surface of the insertion portion.

11. The electrode-equipped endoscope system according to claim 1, which is disposable.

12. The electrode-equipped endoscope system according to claim 1, which is for single use.

13. The electrode-equipped endoscope system according to claim 1, wherein
the insertion portion is inserted into a bladder, and
the bending section is bent such that a distal tip surface of the insertion portion faces a lesion near a communication port with a urethra in the bladder.
